(19) United States
(12) Patent Application Publication
(10) Pub. No.: US 2024/0350694 A1
(43) Pub. Date: Oct. 24, 2024

(54) OZONE TREATMENT FOR CONDITIONED SPACES

(71) Applicant: Preservation Tech, LLC, Hull, IA (US)

(72) Inventors: Nardone Michael, Hull, IA (US); Gonzales Orlando, Hull, IA (US)

(21) Appl. No.: 18/642,113

(22) Filed: Apr. 22, 2024

Related U.S. Application Data
(60) Provisional application No. 63/460,804, filed on Apr. 20, 2023.
(60) Provisional application No. 63/524,623, filed on Jun. 30, 2023.

(51) Int. Cl. A61L 9/12

(52) U.S. Cl. CPC A61L 9/122; A61L 2202/11; A61L 2209/15; A61L 2209/212

(57) ABSTRACT

Disclosed herein is a device, and related systems and methods, designed to increase the shelf-life of products within a confined space through the release of ozone gas. In certain implementations, the device, systems, and methods may be constructed and arranged to power the emission of ozone gas by drawing on wind power of an already-present fan or cooling system.

### TECHNICAL FIELD

The disclosed technology relates generally to ozone generation.

### BACKGROUND

Ozone has long been used as a broad-spectrum biocide against microorganisms. The triatomic form of ozone and its ability to oxidize microorganisms has been applied as a disinfectant in many settings, including, for example, to treat water, kill food bacteria, clean laundry and other fabrics, decontaminate hospital operating rooms, and deodorize air and objects after, for example, a fire.

Despite the long use of ozone as a biocide, there remains a need in the art to improve systems, devices, and methods for generating ozone.

### BRIEF DESCRIPTION OF THE DRAWINGS

FIG. 1 is a partial perspective view of a refrigerated shipping container with an ozone treatment system according to an implementation.

FIG. 2 is a perspective view of an electric generator according to an implementation.

FIG. 3 is a flow diagram of a method for generating gaseous ozone in a conditioned space according to an implementation.

FIG. 4 is a perspective view of an ozone treatment device according to an implementation.

FIG. 5 is an exploded view of the ozone treatment device of FIG. 4.

FIGS. 6A-6D are various views of the ozone treatment device of FIG. 4.

FIG. 7A is a partial perspective view of a refrigerated shipping container with an ozone treatment system with two ozone treatment devices according to an implementation.

FIG. 7B is a close-up view of the ozone treatment system of FIG. 7A.

FIGS. 8A and 8B are perspective and side views of an ozone treatment device according to an implementation.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### DETAILED DESCRIPTION

The disclosed technology enables the generation of gaseous ozone in an enclosed space to, for example, sanitize or disinfect the space. According to one aspect of the disclosure, an ozone treatment system 100 is electrically isolated from the enclosed space and indirectly powered by an airflow system supplying air to the space. The ozone treatment system 100 includes an ozone generator electrically coupled with a wind-driven electric generator. The electric generator is placed in an outflow path of an air supply vent of the enclosed space. The ozone treatment system 100 optionally includes energy storage electrically coupled to the electric generator and the ozone generator. In such cases the energy storage provides a temporary power source for the ozone generator to draw from when, for example, the electric generator is not operating or when the electric generator is producing less electricity than during normal operation.

According to another aspect of the disclosure, a method for generating gaseous ozone in an enclosed space includes generating electricity with a wind-driven electric generator placed in an outflow path of an air supply vent of the enclosed space. The method further includes using the electricity to generate ozone in the enclosed space. The method optionally includes storing electricity produced by the electric generator and then using the stored electricity to generate ozone in the enclosed space when the electric generator is producing less electricity than during normal operation or when the electric generator is not operating. In various implementations the method includes using the stored electricity to generate ozone when there is no outflow from the air supply vent.

The ozone treatment system can be installed in and generate gaseous ozone within a wide variety of enclosed spaces having an airflow system. In various cases the airflow system is a forced air heating, cooling, or other air conditioning system. In such cases the enclosed space can be within any type of room, building, or other space in which the inflow and outflow of air is controlled. In various implementations the enclosed space is within a shipping container. As an example, in such implementations the enclosed space can be the interior of a refrigerated shipping container with a dedicated cooling system that keeps the interior suitable for storing and/or transporting food items such as produce and meat.

Maintaining a level of gaseous ozone within a shipping container is an effective way to extend the shelf life of foodstuffs transported in containers, particularly for ocean-transported containers that have relatively long travel times. Although refrigerated containers and other containers with airflow systems may typically provide an available source of electricity, implementations of the disclosed technology do not directly draw upon the container's electrical capacity. Instead, implementations of the disclosed ozone treatment system include an electric generator driven by a wind turbine, which in turn is driven by the airflow supplied by the container's air conditioning system. Accordingly, the ozone treatment system avoids presenting a direct load on the container's electrical capacity.

Various implementations of the disclosed technology provide one or more additional features and/or benefits. As an example, in various implementations the ozone treatment system can be readily installed within a shipping container (or other enclosed space). In such cases the ozone treatment system can be easily installed since the ozone treatment system does not require integration into the shipping container's onboard wiring, which makes installation straight-forward and uncomplicated. In addition, apart from the occupied space and any needed mounting, implementations of the ozone treatment system are self-contained.

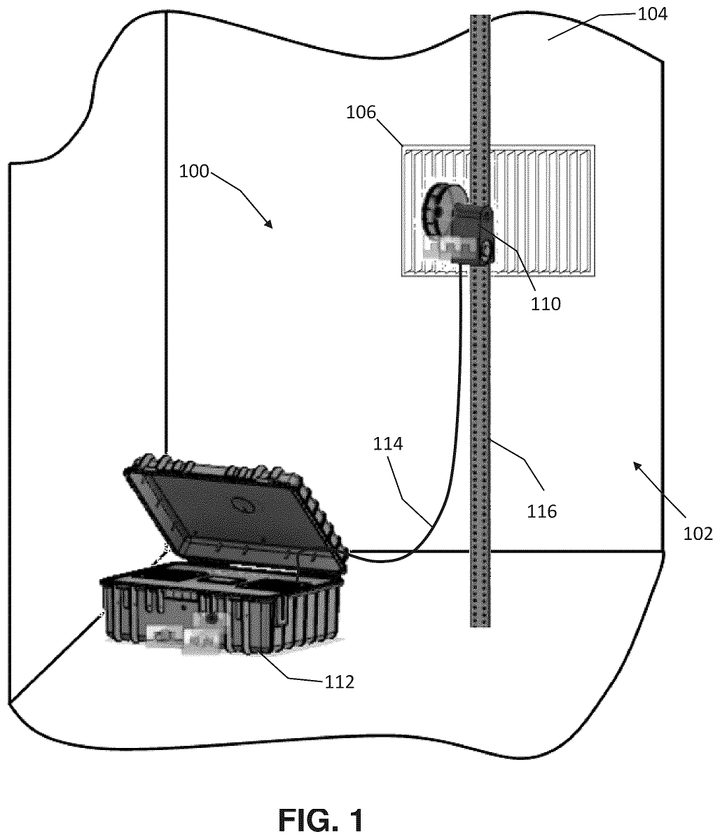

Turning to the drawings, FIG. 1 is a partial perspective view of an ozone treatment system 100 installed within an enclosed space 102 according to various implementations. In this example, the enclosed space 102 is the interior of a refrigerated shipping container 104 that includes an refrigeration system (not shown) for conditioning or refrigerating the interior 102. The shipping container 104 includes an air supply vent 106 that is connected to the refrigeration system for blowing conditioned air into the enclosed space 102. As would be understood, enclosed spaces 102 of different construction, such as HVAC ducts or enclosed rooms, may have alternatives to an air supply vent 106 that provide air flow with alternative methods.

According to the illustrated implementation, the ozone treatment system 100 includes an electric generator 110 that is electrically coupled with an ozone generator 112 via an electrical wire or cable 114.

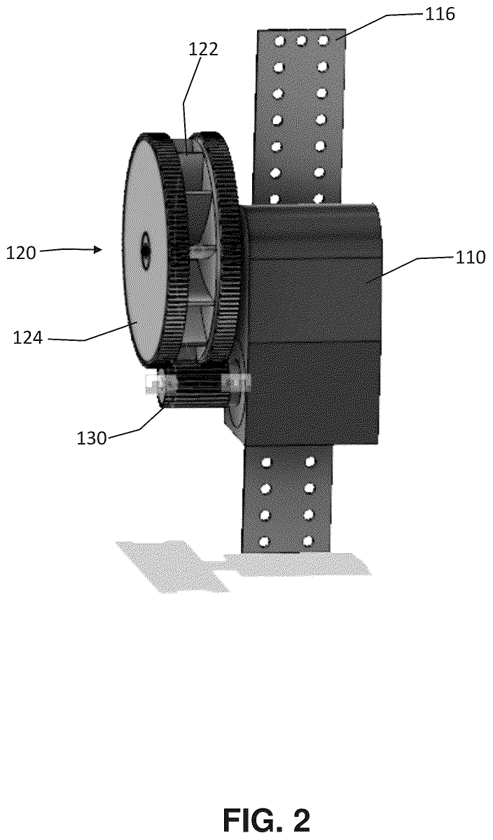

FIG. 2 is a perspective view of the electric generator 110 according to an implementation. The electric generator 110 is mounted to a support beam 116 or other suitable attachment point in an outflow path of the air supply vent 106. As shown in the drawings, the electric generator 110 includes a wind turbine 120 designed to catch the air blown from the supply vent 106. The turbine 120 includes multiple blades 122 positioned about the center of a wheel 124, thus enabling the turbine 120 to convert kinetic energy from the air blown out of the supply vent 106 into mechanical rotation of the wheel 124. The electric generator 110 includes a stator (not shown) and a rotor (not shown) that is rotationally coupled to the wheel 124 that works together with the stator to generate an electric current in response to rotation of the wheel 124.

According to various implementations, one or more portions of the ozone treatment system 100 may be manufactured with a 3D printer. For example, in some cases the wind turbine wheel 124 is a 3D printed part. Of course, other manufacturing methods are possible, such as but not limited to injection molding, casting, welding, stamping, riveting, and any combination thereof.

According to various implementations, the rotational coupling between the wheel 124 and the electric generator's rotor may include one or more gear reductions. In the depicted example, the gearing includes a second, smaller gear 130 that spins more quickly than the wheel 124 in order to increase the rotational speed of the electric generator compared to the rotation of the wheel 124. In various implementations the gearing ratio between the wheel 124 and the electric generator 110 may be set using one or more factors including, but not limited to, for example, the size and/or speed of the wheel 124, the number of blades 122, the speed of the airflow from the supply vent, and the rotational speed needed by the electric generator to produce electricity.

Continuing with reference to FIG. 1, in various implementations the electric generator 110 produces DC power that is delivered to the ozone generator 112 via cable or wire 114. In such cases the ozone generator 112 is configured to accept the DC power, which it uses to run an ozone generating cell. The electrical connection 114 can be provided by any type of suitable electrical cable or wire. In some implementations the cable 114 is an ethernet networking cable that connects to ethernet jacks included with each of the electric generator 110 and the ozone generator 112. In such cases the electric generator 110 is configured to send power to the ozone generator 112 through, for example, the Power over Ethernet (PoE) system. Of course, other wire configurations are possible, such as standard shielded or coated wire of various materials of construction, like copper or aluminum.

In various implementations the ozone treatment system 100 includes one or more sensors that are configured to sense one or more characteristics of the air within the enclosed space and/or the operation of the ozone treatment system. Examples include, but are not limited to, the level of ozone, the temperature, and the humidity of the enclosed space, the ozone production of the ozone generator 110, the turbine speed and electrical power production. In various implementations one, two, or more sensors may be located within the ozone generator 112, within the electric generator 110, and/or within another part of the ozone treatment system 100.

In various implementations the ozone treatment system 100 includes a wireless transmitter (e.g., a radio), or a wired communication port, and is configured to transmit or communicate information about the monitored characteristics to an external system interface (e.g., a software application running on a remove computing device). In some cases, the treatment system 100 is optionally configured to receive communications from an external system interface via a wired and/or wireless communication port.

According to various implementations, the ozone treatment system 100 optionally includes at least one energy storage device (not shown) electrically coupled to the electric generator 110 and to the ozone generator 112. As one possible example, in some cases the ozone treatment system 100 includes one or more rechargeable batteries. As another example, in some cases the ozone treatment system includes one or more capacitors such as, for example, as part of a supercapacitor bank. In such cases the energy storage device can temporarily power the ozone generator when the electric generator is not operating or when the electric generator is producing less electricity than during normal operation. As an example, in some cases the refrigeration system of a refrigerated shipping container may cycle on and off depending on a particular cooling profile and the temperature of the interior of the container. In various cases, the at least one energy storage device enables the ozone treatment system 100 to continue generating ozone when the refrigeration system has been turned off.

As shown in FIG. 1, the electric generator 110 can be mounted to a support beam 116 in an outflow path of the air supply vent 106. Depending upon the internal structure of the enclosed space 102, the electric generator 110 may be mounted to any suitable attachment point within the enclosed space 102 so that the electric generator 110 is positioned within the outflow path of the air supply vent 106. In addition, attachment or mounting points are not limited to pre-existing structure within the enclosed space (e.g., structural support members), but may include a frame or other mounting structure specially installed with the electric generator 110.

According to various implementations, the ozone treatment system 100 includes a control system that operates various aspects of the treatment system. In various cases the control system includes at least one processor that is configured to perform particular operations or actions by virtue of loading and executing instructions stored in one or more memory devices. Parts of the control system, including the processor and at least one memory device, can be located in the ozone generator 112, the electric generator 110, and/or in one or more other portions of the ozone treatment system 100, including within a dedicated housing. In some cases the control system may receive and/or transmit information over the power cable 114 between the ozone generator 112 and the electric generator 110.

The processor carrying out the instructions causes the control system, the ozone generator 112, the electric generator 110, and/or another portion of the treatment system 100 to carry out the desired actions. For example, in various cases the control system is configured to receive environmental data from one or more sensors and then communicate relevant information about the data to an external system interface. In some cases, the control system may receive instructions from a remote system interface and carry out one or more actions based on those instructions. In some cases, the control system may be configured to determine a level of gaseous ozone in the enclosed space based on sensor data and then adjust the time period or rate of ozone production based on the determined ozone level.

According to various implementations, the processor is a Central Processing Unit (CPU), although other processors such as, but not limited to, microprocessors, microcontrollers, Field Programmable Gate Arrays (FPGAs), and Application Specific Integrated Circuits (ASICs) are possible. In various cases the processor includes or is coupled with one or more physical, non-transitory computer accessible or readable storage devices, which are also referred to herein as “memory” and “memory devices.” The memory may be implemented using any suitable memory technology, which may include, e.g., temporary and more long-term configurations, volatile and non-volatile configurations, and solid state and/or other physical formats. Examples of possible memory include random access memory (RAM), dynamic random-access memory (DRAM), static random-access memory (SRAM), magnetic hard discs, optical discs, floppy discs, flash memory, forms of electrically programmable memory (EPROM) and electrically erasable and programmable (EEPROM) memory, and other forms known in the art.

According to various implementations, one or more memory device(s) coupled with the processor contain instructions for configuring the processor to perform particular operations or actions by virtue of loading and executing the instructions. The processor carrying out the instructions causes the control system and/or the ozone treatment system 100 to carry out the desired actions. References herein to the processor and/or treatment system 100 carrying out various activities imply that the processor is configured with corresponding instructions for execution.

According to various implementations, an example of the ozone treatment system 100 includes the electric generator 110 electrically coupled with the ozone generator 112 through an Ethernet networking cable 114. The ozone treatment system 100 is mounted within the enclosed space 102 of a refrigerated shipping container 104. In various cases the shipping container is connected with a 480 V line voltage from an ocean-going ship, with which the shipping container 104 operates its own refrigeration system. For example, the refrigeration system may be configured to maintain a temperature of between 0-5 degrees Celsius by blowing refrigerated air into the shipping container's interior and enclosed space 102 through at least one air supply vent 106. In some cases, the container blower may introduce conditioned air through the vent 106 at a rate of about 2,000 cfm.

According to this example, the ozone treatment system 100 is configured to produce electricity with the wind-driven electric generator 110 based on a blower air supply of at least 1,200 cfm. In some cases, the wind turbine 120 uses a 5:1 gear reduction. This gear ratio enables the turbine to spin the generator at about 3,500 rpm, assuming that the conditioned air flow from the supply vent 106 rotates the turbine wheel at about 700 rpm. In some cases, the turbine wheel 124 includes more than seven blades 122. Examples include, but are not limited to, nine blades 122 and twelve blades 122.

In such a case, the ozone generator 112 is configured to operate with an input power supply of 12 VDC and generate about 1 gram of ozone per hour. In some cases, the ozone treatment system 100 includes an energy storage system that includes a supercapacitor bank providing about 125 F (farad) of capacitance at 12 VDC. In some cases, the system 100 may use a smaller capacitor bank that provides a capacitance of 1.5 F.

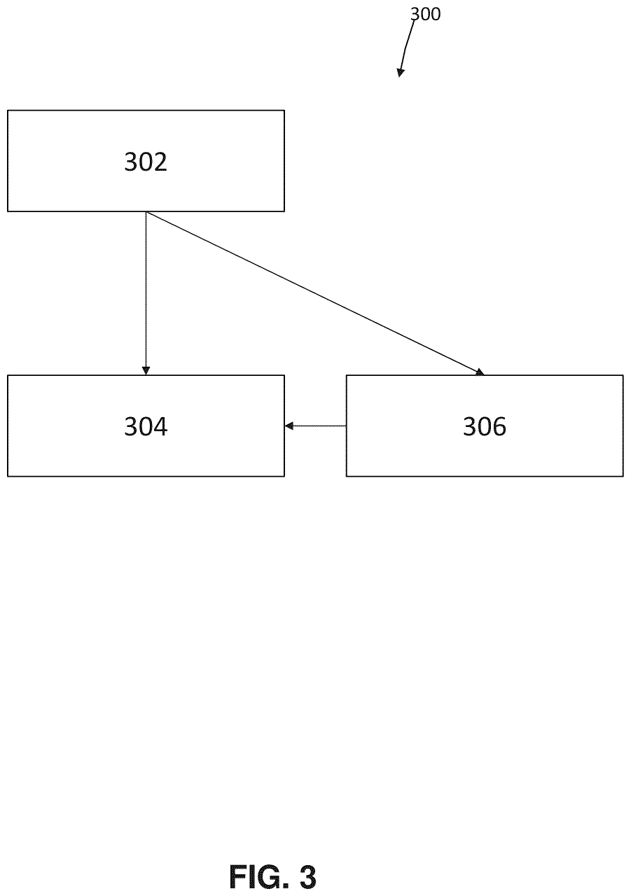

FIG. 3 is a flow diagram of a method 300 for generating gaseous ozone in a conditioned space according to an implementation. The method 300 for generating gaseous ozone in an enclosed space includes generating electricity 302 with a wind-driven electric generator placed in an outflow path of an air supply vent of the enclosed space. The method further includes using the electricity to generate ozone 304 in the enclosed space. The method optionally includes storing electricity 306 produced by the electric generator and then using the stored electricity to generate ozone 304 in the enclosed space when the electric generator is producing less electricity than during normal operation or when the electric generator is not operating. In various implementations the method includes using the stored electricity to generate ozone when there is no outflow from the air supply vent. In some cases, the method 300 includes generating gaseous ozone within the interior of a shipping container, such as a refrigerated shipping container.

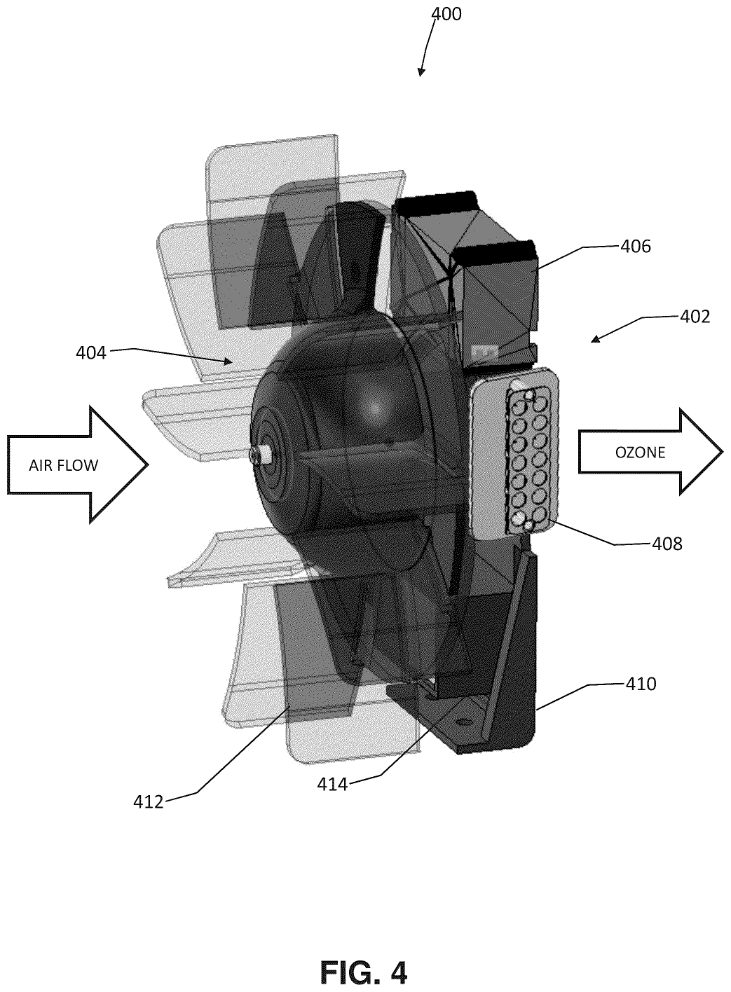

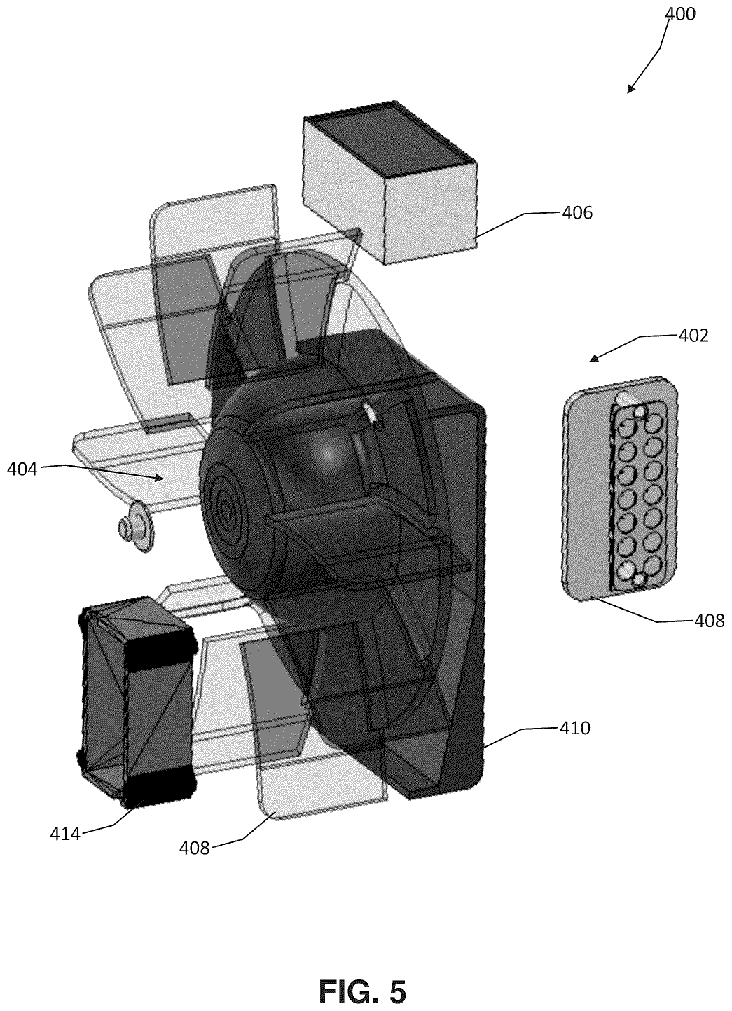

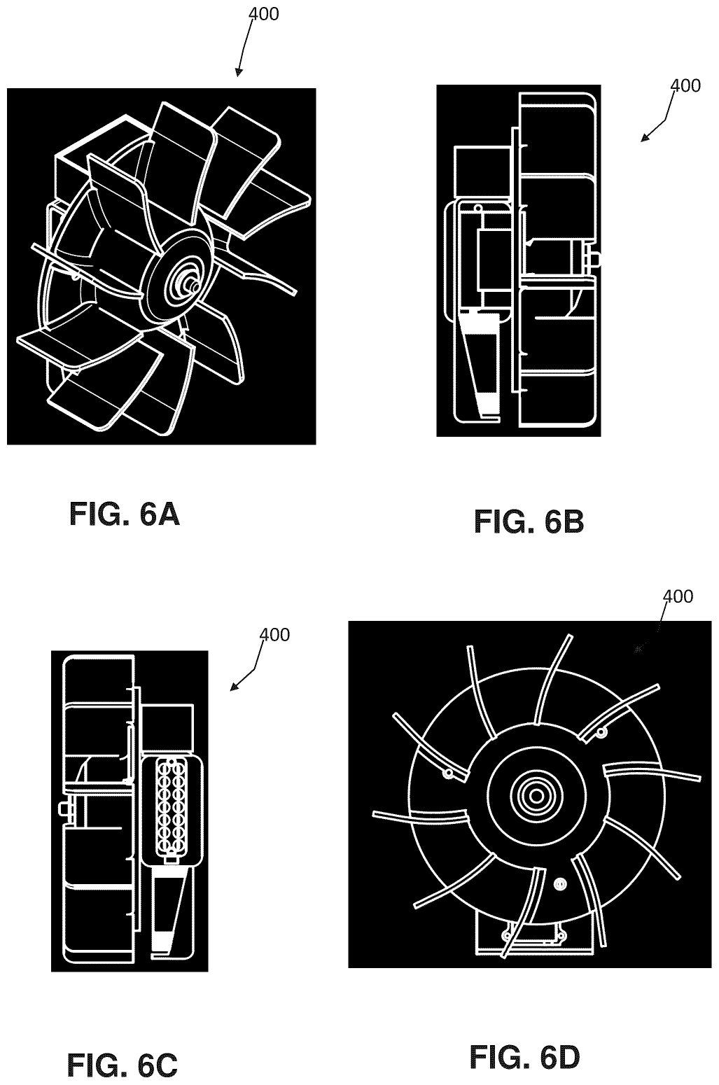

Turning now to FIGS. 4A-6D, according to another aspect of the disclosure, an ozone treatment device 400 includes an ozone generator 402 mounted together with a wind-driven electric generator 404 to a common frame 406. The device 400 may also be referred to as an ozone treatment system 400 and various aspects, features, and details previously described with respect to the examples in FIGS. 1-3 also apply to the device 400 as appropriate and as will be appreciated from the descriptions, drawings and various contexts. The device is configured to generate ozone while being electrically isolated from the enclosed space and indirectly powered by an airflow system supplying air to the space. The ozone generator 402 mounted to the frame 406 is electrically connected with the electric generator 404. The treatment device 400 is placed in an outflow path of an air supply vent of the enclosed space. The ozone treatment device 400 optionally includes energy storage electrically coupled to the electric generator and the ozone generator. In such cases the energy storage provides a temporary power source for the ozone generator to draw from when, for example, the electric generator is not operating or when the electric generator is producing less electricity than during normal operation.

The ozone treatment device 400 can be installed in and generate gaseous ozone within a wide variety of enclosed spaces having an airflow system. In various cases, the airflow system is a forced air heating, cooling, or other air conditioning system. In such cases, the enclosed space can be within any type of room, building, or other space in which the inflow and outflow of air is controlled. In various implementations the enclosed space is within a shipping container. As an example, in such implementations the enclosed space can be the interior of a refrigerated shipping container with a dedicated cooling system that keeps the interior suitable for storing and/or transporting food items such as produce and meat.

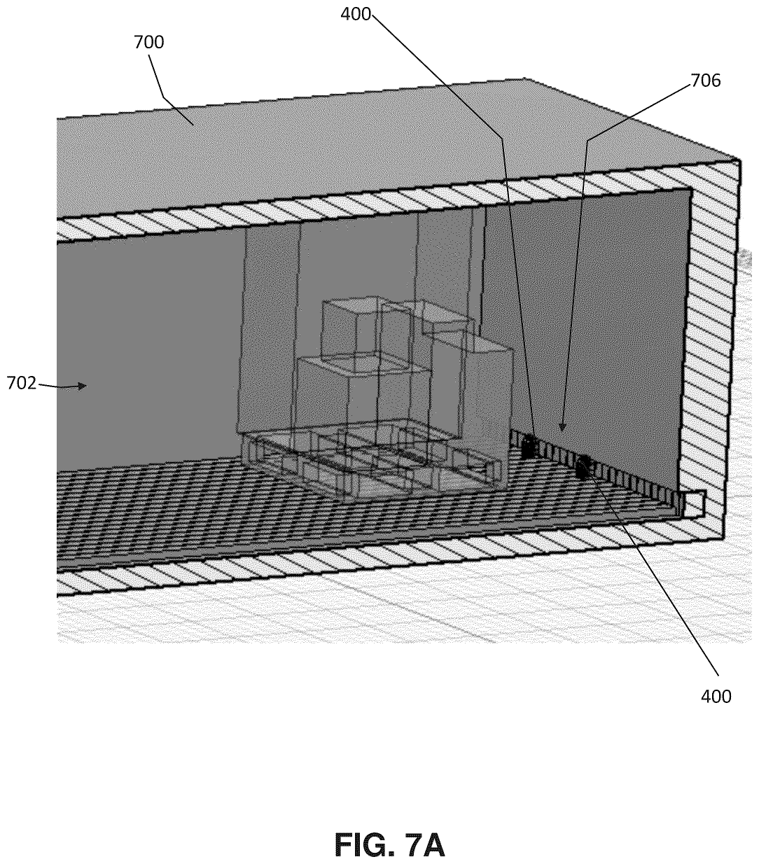

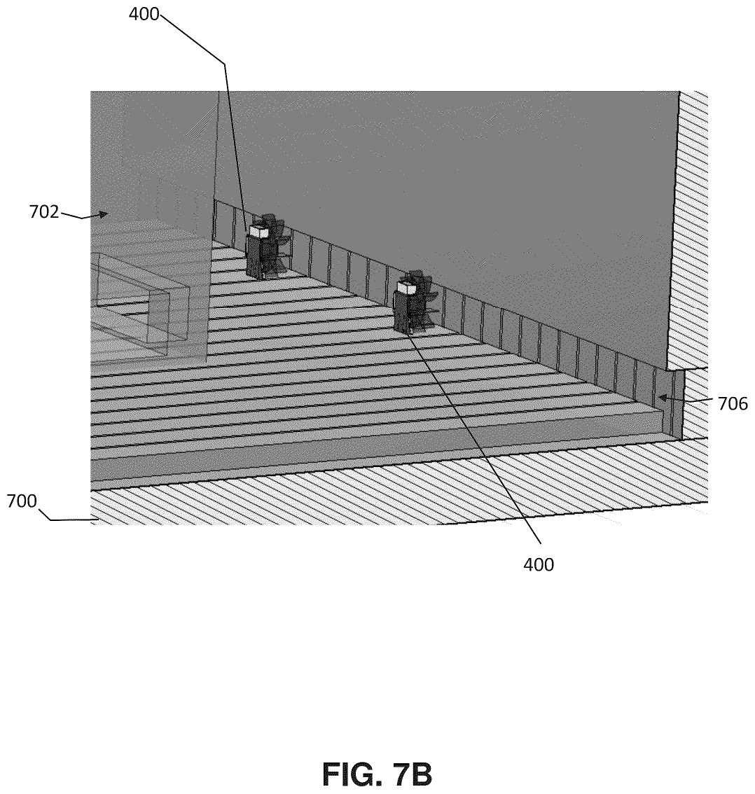

FIGS. 7A and 7B are partial views of a shipping container 700 with two ozone treatment devices 400 installed for form an ozone treatment system in the enclosed space 702 of the interior of the container 700.

Maintaining a level of gaseous ozone within a shipping container is an effective way to extend the shelf life of foodstuffs transported in containers, particularly for ocean-transported containers that have relatively long travel times. Although refrigerated containers and other containers with airflow systems may typically provide an available source of electricity, implementations of the disclosed technology do not directly draw upon the container's electrical capacity. Instead, implementations of the disclosed ozone treatment device include an electric generator driven by a wind turbine, which in turn is driven by the airflow supplied by the container's air conditioning system. Accordingly, the ozone treatment device avoids presenting a direct load on the container's electrical capacity.

Various implementations of the disclosed technology provide one or more additional features and/or benefits. As an example, in various implementations the ozone treatment device can be readily installed within a shipping container (or other enclosed space). In such cases the ozone treatment device can be easily installed since the ozone treatment device does not require integration into the shipping container's onboard wiring, which makes installation straight-forward and uncomplicated. In addition, apart from the occupied space and any needed mounting, implementations of the ozone treatment system are self-contained.

As with the system in FIG. 1, the ozone treatment device 400 in FIGS. 4-6D can be installed within an enclosed space 702 according to various implementations. In various examples, the enclosed space 702 is the interior of a refrigerated shipping container 700 that includes a refrigeration system for conditioning or refrigerating the interior. The shipping container includes an air supply vent 706 that is connected to the refrigeration system for blowing conditioned air into the enclosed space. The air supply vent may be located in various positions along one or more walls of the container. In various cases two or more air supply vents may be present on the same and/or different container walls. In some cases an air supply vent 706 may run along the width and/or length of the container 700, for example, next to the floor or ceiling of the container.

As shown in the drawings, the ozone treatment device 400 includes an electric generator 404 that is electrically coupled with an ozone generator 402 via a suitable electrical wire, cable, connector or the like. The ozone generator 402 in this example includes a step up transformer 406 electrically coupled with an ozone corona discharge plate 408. The electric generator 404 and ozone generator 402 are mounted to a support frame 410 that can be attached or fixedly mounted to a suitable attachment point in an outflow path of a container's air supply vent. As shown in the drawings, the electric generator 404 includes a wind turbine 412 designed to catch the air blown from the supply vent. In this case the turbine 412 has a designed axial air flow. The turbine 412 includes multiple blades positioned about the center, thus enabling the turbine 412 to convert kinetic energy from the air blown out of the supply vent into mechanical rotation of a rotor (not shown). The rotor works together with a stator (not shown) inside an electronics housing behind the turbine to generate an electric current in response to rotation of the turbine 412. Conditioning circuitry 414 conditions the electrical current before it reaches transformer 406 and ozone plate 408.

According to various implementations, one or more portions of the ozone treatment system 100 may be manufactured with a 3D printer. For example, in some cases the wind turbine 412 is a 3D printed part. According to various implementations, the rotational coupling between the turbine 412 and the electric generator's rotor may include one or more gear reductions. In various implementations the wind turbine 412 may be directly coupled to the rotor as in the example shown in FIGS. 4-6D.

In various implementations the ozone treatment device 400 includes one or more sensors that are configured to sense one or more characteristics of the air within the enclosed space and/or the operation of the ozone treatment system. Examples include, but are not limited to, the level of ozone, the temperature, and the humidity of the enclosed space, the ozone production of the ozone generator 402, the turbine speed and electrical power production. In various implementations one, two, or more sensors may be located within the ozone generator 402, within the electric generator 404, and/or within another part of the ozone treatment device 400.

In various implementations the ozone treatment device 400 includes a wireless transmitter (e.g., a radio) or a wired communication port and is configured to transmit or communicate information about the monitored characteristics to an external system interface (e.g., a software application running on a remove computing device). In some cases, the treatment device 400 is optionally configured to receive communications from an external system interface via a wired and/or wireless communication port.

According to various implementations, the ozone treatment device 400 optionally includes at least one energy storage device (not shown) electrically coupled to the electric generator 404 and to the ozone generator 402. As one possible example, in some cases the ozone treatment device 400 includes one or more rechargeable batteries. As another example, in some cases the ozone treatment device 400 includes one or more capacitors such as, for example, as part of a supercapacitor bank. In such cases the energy storage device can temporarily power the ozone generator when the electric generator is not operating or when the electric generator is producing less electricity than during normal operation. As an example, in some cases the refrigeration system of a refrigerated shipping container may cycle on and off depending on a particular cooling profile and the temperature of the interior of the container. In various cases, the at least one energy storage device enables the ozone treatment device 400 to continue generating ozone when the refrigeration system has been turned off.

According to various implementations, the ozone treatment device 400 can be mounted to any suitable attachment point within a shipping container as long as the mounting point allows the outflow from one or more container vents to reach the wind turbine. As shown in FIGS. 7A-7B, in various cases in which the air vent 706 is located close to the floor and the treatment device 400 may be mounted to the floor (e.g., bolted through attachment holes on the bottom of the frame) directly in front of the vent 706. In the illustrated example, two treatment devices 400 are mounted to the floor in front of the vent 706.

According to various implementations, the ozone treatment device 400 includes a control system that operates various aspects of the treatment system. In various cases the control system includes at least one processor that is configured to perform particular operations or actions by virtue of loading and executing instructions stored in one or more memory devices. Parts of the control system, including the processor and at least one memory device, can be located in the ozone generator 402, the electric generator 404, and/or in one or more other portions of the ozone treatment device 400, including within a dedicated housing.

The processor carrying out the instructions causes the control system, the ozone generator 402, the electric generator 404, and/or another portion of the treatment device 400 to carry out the desired actions. For example, in various cases the control system is configured to receive environmental data from one or more sensors and then communicate relevant information about the data to an external system interface. In some cases, the control system may receive instructions from a remote system interface and carry out one or more actions based on those instructions. In some cases, the control system may be configured to determine a level of gaseous ozone in the enclosed space based on sensor data and then adjust the time period or rate of ozone production based on the determined ozone level.

According to various implementations, the processor is a Central Processing Unit (CPU), although other processors such as, but not limited to, microprocessors, microcontrollers, Field Programmable Gate Arrays (FPGAs), and Application Specific Integrated Circuits (ASICs) are possible. In various cases the processor includes or is coupled with one or more physical, non-transitory computer accessible or readable storage devices, which are also referred to herein as “memory” and “memory devices.” The memory may be implemented using any suitable memory technology, which may include, e.g., temporary and more long-term configurations, volatile and non-volatile configurations, and solid state and/or other physical formats. Examples of possible memory include random access memory (RAM), dynamic random-access memory (DRAM), static random-access memory (SRAM), magnetic hard discs, optical discs, floppy discs, flash memory, forms of electrically programmable memory (EPROM) and electrically erasable and programmable (EEPROM) memory, and other forms known in the art.

According to various implementations, one or more memory device(s) coupled with the processor contain instructions for configuring the processor to perform particular operations or actions by virtue of loading and executing the instructions. The processor carrying out the instructions causes the control system and/or the ozone treatment device 400 to carry out the desired actions. References herein to the processor and/or treatment device 400 carrying out various activities imply that the processor is configured with corresponding instructions for execution.

According to various implementations, an example of the ozone treatment device 400 includes the electric generator 404 electrically coupled with the ozone generator 402 through one or more cable connectors. The ozone treatment device 400 is mounted within the enclosed space of a refrigerated shipping container. In various cases, two or more ozone treatment devices 400 are mounted within the shipping container 700 as shown in FIGS. 7A-7B. For example, one device may be mounted in front of a first vent and another device may be mounted in front of a second vent. As another example, multiple devices 400 may be mounted in front of a single, larger (e.g., wider) vent. In various cases the shipping container 700 is connected with a 480 V line voltage from an ocean-going ship, with which the shipping container operates its own refrigeration system. For example, the refrigeration system may be configured to maintain a temperature of between 0-5 degrees Celsius by blowing refrigerated air into the shipping container's interior and enclosed space through at least one air supply vent.

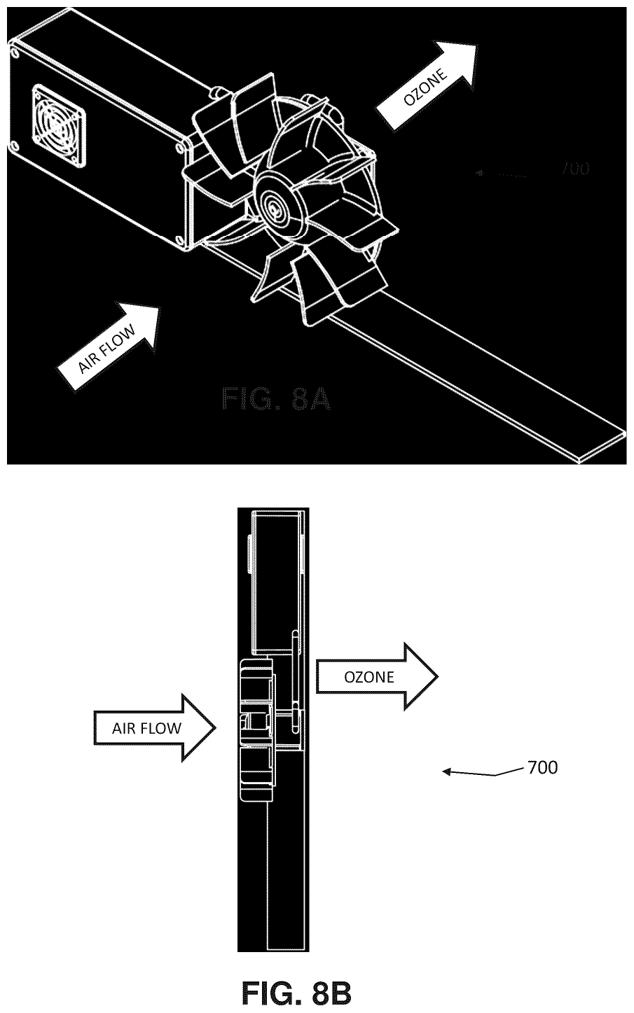

FIGS. 8A and 8B illustrate another example of an ozone treatment device 800 according to various implementations. The various features, aspects and details describing other examples herein likewise apply to the ozone treatment device 800 as will be appreciated from the drawings.

Although the disclosure has been described with reference to preferred embodiments, persons skilled in the art will recognize that changes may be made in form and detail without departing from the spirit and scope of the disclosed apparatus, systems and methods.

What is claimed is:

1. An ozone treatment device and/or system, comprising:
an electric generator comprising a wind turbine; and
an ozone generator electrically coupled with the electric generator.

2. The ozone treatment device and/or system of claim 1, further comprising an energy storage device electrically coupled with the electric generator and the ozone generator.

3. The ozone treatment device and/or system of claim 1, further comprising a mounting frame, wherein the electrical generator and the ozone generator are mounted to the frame adjacent to one another.

4. A method for generating gaseous ozone in an enclosed space, comprising:
generating electricity with a wind-driven electric generator placed in an outflow path of an air supply vent of the enclosed space; and
using the electricity to generate ozone in the enclosed space.

5. The method of claim 4, further comprising storing electricity produced by the electric generator and then using the stored electricity to generate ozone in the enclosed space when the electric generator is producing less electricity than during normal operation or when the electric generator is not operating.
